(19) United States
(12) Patent Application Publication
(10) Pub. No.: US 2009/0170123 A1
(43) Pub. Date: Jul. 2, 2009

(54) Identification of Novel Protein Targets on the Surface of Stressed Cells

(76) Inventors: Fernando Donate, San Diego, CA (US); Jose C. Juarez, San Diego, CA (US); Andrew P. Mazar, Highland Park, IL (US)

Correspondence Address: BROWDY AND NEIMARK, P.L.L.C.;624 NINTH STREET, NW, SUITE 300, WASHINGTON, DC, 20001-5303, US

(21) Appl. No.: 12/091,003

(22) PCT Filed: Oct. 23, 2006
(86) PCT No.: PCT/US2006/041285

§ 371 Date: Sep. 3, 2008

Related U.S. Application Data
(60) Provisional application No. 60/728,757, filed on Oct. 21, 2005.

(51) Int. Cl. G01N 33/53; C12Q 1/02

(52) U.S. Cl. 435/7.2; 435/29

(57) ABSTRACT

The present invention in the field of biochemistry and medicine is directed to novel methods for identifying molecules, typically proteins, that move to the cell surface when cells are stimulated or stressed can act as receptors even thought they are not transmembrane molecules and normally originate in the cytosol. Such molecules are useful targets for development of agents that can image or treat tumors or other pathologies. Methods to detect or identify such proteins that have translocated to the cell surface when cells are stressed by an angiogenic environment, environmental stresses, the stimulation of cell proliferation and differentiation, or after exposure to certain drugs such as cancer chemotherapeutics, are disclosed.

### BACKGROUND OF THE INVENTION

1. Field of the Invention

The present invention in the field of biochemistry and medicine is directed to novel methods for identifying molecules that move to the cell surfaces under certain conditions and act as receptors even thought they are not transmembrane molecules. Such molecules serve as useful targets for development of agents that can image or treat tumors or other pathologies.

2. Description of the Background Art

In endothelial cells (ECs) as well as other types of cells, including tumor cells, proteins can be induced to translocate to the cell membrane under certain conditions such as angiogenic stimulation and/or stress. These proteins are normally in a different cellular compartment, such as the cytoplasm. Thus, they may lack the transmembrane domains which are generally present in membrane proteins, at least those that themselves are anchored in, and span, the membrane. Proteins lacking such typical linkages to the plasma membrane, these translocatable proteins, may nevertheless associate with other molecules that are anchored in, or to, the membrane.

### Externalization of Proteins as Part of the Stress Response

This has recently been reviewed by several of the present inventors (Donãte F et al., Curr Cancer Drug Targets, 2004, 4:543-53). The externalization of intracellular proteins (to the outside of the cell) has been observed with other proteins in addition to tropomyosin (Tpm). These externalized proteins frequently behave as receptors for plasma and other extracellular proteins and enzymes, and in this capacity, regulate a number of extracellular events. The biological activities that are regulated by these proteins are often in response to cellular challenge or stress (angiogenesis may be considered a stress response since it occurs in response to a physiological challenge, for example, in wound healing or when cancer is present), and we have hypothesized that the externalization of intracellular proteins, which then regulate extracellular functions, may be part of a general stress response. For example, several studies have suggested that the Tpm binding protein, actin, can also be externalized to the surface of ECs (Dudani, A K et al., Br. J. Haematol. 1996, 95:168-78), and externalized actin may act as a receptor for the proangiogenic molecule angiogenin as well as for plasminogen (Dudani et al, supra). The kininogen-binding and complement-activating protein p33/gC1qR was observed immunohistochemically to be predominantly intracellular, suggesting that it first had to be externalized before being capable of assembling the components of the kinin-forming and complement-activating pathways (Dedio, J et al.; FEBS Lett. 1996, 399:255-58). The F1Fo ATPase, normally found in the mitochondria, has also been localized to the cell surface and appears to be an antiangiogenic receptor for angiostatin (Moser, TL et al., Proc. Natl. Acad. Sci. USA 1999, 96:2811-16; Moser, TL et al.; Proc. Natl. Acad. Sci. USA 2001, 98:6656-61).

Several broader classes of proteins have also been demonstrated to be externalized. Annexins I, II and V are localized to the cytoplasm in quiescent cells (Jans, SW et al.; J. Mol. Cell Cardiol. 1995, 27:335-48; Singh, A K, Curr. Opin. Nephrol. Hypertens. 2001:10; Kwaan, H C et al.; Hematol. Oncol. Clin. North Am. 2003, 17:103-114; Kim, J et al; Front. Biosci. 2002, 7:d341-d348; Tuszynski, GP et al., Microvasc. Res. 2002, 64:448-62; Chapman, L et al.; Endocrinology 2002, 143:4330-38; Solito, E et al.; J. Immunol. 2000, 165:1573-81). Externalized annexin V in ECs may protect them from exposure to thrombogenic phospholipids. The immunopathogenesis of the anti-phospholipid antibody syndrome may involve displacement of Annexin V from the EC surface by anti-phospholipid antibodies (Singh, supra). Annexin II is also externalized by ECs and other cell types, and regulates cell surface fibrinolysis by binding t-PA and plasminogen (Kwaan et al., supra; Kim et al., supra)]. Similar to annexin V, annexin II has been implicated in the immunopathogenesis of the anti-phospholipid antibody syndrome, where antibodies against annexin II have been observed at higher levels in patients than in normal controls (Kwaan et al., supra). Angiostatin also binds to annexin II, implicating this protein in the regulation of angiogenesis (Tuszynski et al., supra). Finally, annexin I is externalized by multiple cell types including monocytes and folliculo-stellate cells (Chapman et al., Solito et al., supra). Annexin I regulates inflammatory responses by interfering with leukocyte-endothelial adhesive events and through the regulation of steroid signaling.

Several members of the heat shock protein family are also externalized and interact with cells of the immune system, exerting an immunoregulatory role. HSP70 increases the expression of pro-inflammatory cytokines in human monocytes, a signaling effect that is transduced by the Toll-like receptors TLR2 and TLR4 (Asea, A et al.; J. Biol. Chem. 2002, 277:15028-34). In addition to the direct signaling effects of apo-HSP70, HSP70-peptide complexes are taken up through a CD40-dependant pathway on monocytes and dendritic cells, which may serve to prime these cells for immune responses against the bound peptide (or a larger protein the antigen from which the peptide was derived) (Becker, T et al.; J. Cell Biol. 2002, 158:1277-85). Another member of the HSP family that is also normally found in the cytosol, HSP20, regulates platelet function in response to endothelial injury (Kozawa, 0 et al.; Life Sci. 2002, 72:113-124). Finally, GRP78 is an ER resident protein that has been identified on the surface of different cell types (Berger, CL et al.; Int. J. Cancer 1997, 71:1077-85; Triantafilou, M et al., Hum. Immunol, 2001, 62:764-70) and is also a receptor for the kringle 5 domain of plasminogen (K5), an anti-angiogenic protein fragment (Davidson, D J. Pub'd U.S. Patent Applic #2002/11519, 2003).

The present inventors and their colleagues showed that Tpm is translocated to the surface of ECs under angiogenic conditions (Proc. Natl. Acad. Sci. U.S.A. 99; 12224-12229, 2002). See also WO/03/077872. Thus, the discovery of externalized Tpm is believed to fall into a newly evolving paradigm of intracellular protein externalization in response to cellular stress. The present inventors examined the biological significance of this externalization in ECs and have identified Tpm as a common receptor for multiple anti-angiogenic proteins, suggesting that Tpm-dependent anti-angiogenic effects represent a common pathway for the inhibition of angiogenesis.

### SUMMARY OF THE INVENTION

The present invention is directed to a method of detecting or identifying one or more non-membrane proteins that translocate to the surface of stressed or stimulated cells but are not present on the surface of the same cells when these cells are quiescent, the method comprising
(a) treating cells from a biological sample under conditions that remove the translocated protein from the cell surface but preserve the integrity of the cells' membrane,
(b) detecting or identifying proteins removed from the cells surface, thereby detecting or identifying the translocated protein. Step (a) is intended to remove from the cells any non-anchored proteins. In the above methods, translocated proteins are typically cytosolic proteins.

The removing of step (a) is preferably accomplished by acid wash under isotonic conditions to minimize cell lysis using a buffer with a pH range of 1 to 5. The removing may also be accomplished by membrane isolation, proteolysis, high salt treatment or metal chelation.

The translocated protein may be a receptor for a known ligand and the detecting or identifying comprises, prior to step (a), measuring occupancy of the receptors on the surface of the cells using the known ligands in a receptor-ligand binding assay.

Step (b) above preferably comprises a step of comparing proteins removed from the stressed cells with proteins removed under the same conditions from control cells of the same or similar cell type that are not stimulated or stressed. In one embodiment, the comparison is between cells stressed by an angiogenic agent and control cells maintained in a basal medium

The translocated and removed proteins are identified by any of a number of techniques that are well-known in the art such as gel electrophoresis or two-dimensional (2D) analysis using gel electrophoresis and isoelectric focusing. The translocated and removed proteins may be further identified by excision of a spot or spots from 2D gels followed by mass spectroscopy (MS) or by proteolytic digestion with any proteolytic enzyme, a preferred example of which is trypsin or chymotrypsin, most preferably trypsin, combined with mass spectroscopy.

In another embodiment, the excised spot or spots are subjected to proteolytic, preferably tryptic, digestion, western blotting and N-terminal sequencing or amino acid composition analysis.

In another embodiment, the translocated proteins are crosslinked prior to the removing step (a) and are identified by proteolytic digestion and mass spectroscopy.

In one embodiment, the stressed cells are stressed by angiogenic conditions. In other embodiments, they are stressed by environmental stimuli such as hypoxia or oxidative stress. The cells may also be stressed by the processes of proliferation or differentiation. The stressed cells may also result from exposure to a growth factor or cytokine in vivo prior to their preparation or ex vivo. Additional cell stressors include pro-inflammatory stimulus, such as IL-8, or a stimulus that induces NFκB. Another form of cell stress is as a result of exposure to a cancer chemotherapeutic drug or agent.

In a preferred embodiment, the stressed cells are endothelial cells (ECs). In one preferred method, the translocated and removed protein is tropomyosin. Step (a) may be conducted about six hours after initiation of cells are stressed or stimulated. In another embodiment the translocated protein is vimentin. The stressed cells may also be tumor cells, monocyte/macrophages, neutrophils or T lymphocytes, or stem cells or other progenitor cells.

The translocated protein may be one that serves as a receptor for an anti-angiogenic protein such as angiostatin, an example of which is the angiostatin-binding F₁F₀-ATPase subunit.

The antiangiogenic protein may be plasminogen kringle region 5 (K5) and the K5-binding translocated protein is GRP78. The anti-angiogenic protein may be high molecular weight kininogen (HKa), histidine-proline-rich glycoprotein (HPRG) or an annexin. Tpm is another example of a translocated protein that binds to anti-angiogenic proteins.

Also provided is a method of determining whether tumor cells or normal cells of a subject with a tumor are resistant to a chemotherapeutic agent which results in translocation or externalization of internal proteins to the cell surface, comprising subjecting the subject or tumor or normal cells from the subject to chemotherapy with the agent and detecting or identifying proteins removed from the cells surface using the methods described above, wherein the detection of the presence of the translocated proteins is indicative of resistance to chemotherapy by the agent.

The present method described herein may further comprising, after detecting or identifying the translocated protein, a step of (c) isolating the protein.

The invention is also directed to a method of producing an antibody specific for a translocated protein as the protein is expressed on the surface of a stressed cell, comprising performing the above method to isolate the protein, and further, the step of (d) producing an antibody specific for the translocated isolated protein that distinguishes between the translocated protein and the same protein that has not been translocated by the stress.

The invention includes a method for identifying cells that have undergone stress-related translocation of internal proteins to the cell surface comprising contacting cells suspected of having such a translocated protein on its surface with an antibody, such as an antibody produced as above.

Also provided is a method of diagnosing a pathological process in a subject in which cells are stressed or stimulated to translocate internal proteins to the cell surface, comprising detecting or identifying the translocated proteins on a sample of cells from the subject and comparing with control, quiescent cells from the same subject or a control subject not undergoing the pathological process. The pathological process in preferably an angiogenic process.

### BRIEF DESCRIPTION OF THE DRAWINGS

FIG. 1 shows a 2D gel of proteins in an acid wash fraction of HUVEC that had earlier been stimulated by FGF-2 and biotin labeled using biotin-XX (Invitrogen, B6352). Avidin-HRP was used to detect proteins.

FIG. 2 shows a 2D gel of proteins in acid washed control HUVEC (not treated with FGF-2) which have been earlier labeled with biotin using biotin-XX (Invitrogen, B6352). Avidin-HRP was used to detect proteins.

FIG. 3 shows the merged images of FIGS. 1 and 2 demonstrating a different pattern of proteins exposed in the membrane and suitable to be eluted by acid wash in FGF-2-treated vs, non-treated HUVEC.

FIG. 4 shows Western blots of tropomyosin (Tpm) that has been removed from the surface of HUVEC by acid wash (“WCE”: Whole cell extract). HUVEC were subjected to an acid wash, and the wash was precipitated by acetone. Various preparations were electrophoresed, transferred and blotted using polyclonal rabbit anti-tropomyosin antiserum (Sigma T3651) to detect tropomyosin. An actin-specific antibody was also used. Purified chicken gizzard tropomyosin (cgTM), purified human Tpm isoform 5 (h-TM5) and isoform 3 (h-TM3) were used as positive controls. The antibody recognizes all three Tpm proteins.

FIG. 5 shows a quantitation of the time course of Tpm externalization as revealed by an acid wash of HUVEC stimulated with FGF-2. Tpm was identified in the Western blot with the TM311 anti-Tpm monoclonal antibody (mAb).

FIG. 6 shows a quantitative determination of the levels of Tpm externalization as revealed by an acid wash of HUVEC stimulated with FGF-2 in confluent versus non-confluent cultures. Tpm was identified in the Western blot with the TM311 anti-Tpm mAb.

FIGS. 7A and 7B show 2D gels (Western blots) of an acid wash fraction of HUVECs stimulated with FGF-2. The NFκB protein is absent from the acid wash, but present in the whole cell lysate (WCE) demonstrating that the acid wash treatment did not lyse the cells. After the IPG IEF/DTT/iodoacetamide is run at 500 V for 3.5 hours followed by 4-12% NuPAGE in MES in the other dimension according to the manufacturer's instructions (Invitrogen), the blots were probed with anti-NFκB rabbit polyclonal antibody (Santa Cruz, sc-109) at 1/2000 for 2 hrs at room temperature.

FIGS. 8A and 8B show the presence of Tpm protein in whole cell extracts (WCE) and acid wash of HUVEC, reprobing the blots used in FIGS. 7a and 7b with the anti-Tpm mAb TM311 (Sigma; 1/2000 dilution of ascites fluid), overnight at 4° C.

FIGS. 9A and 9B are Western blots showing that vimentin (FIG. 9A), but not the control abundant cytosolic protein, β-tubulin (FIG. 9B), was detected in the acid wash fraction of HUVEC. Blots were probed with the anti-vimentin antibody (goat anti-vimentin (Sigma V4630, 1/2000, overnight incubation) and with a rabbit anti-β-tubulin (Santa Cruz sc9104), 1/1000, for 1 hr.

### DESCRIPTION OF THE INVENTION

The present inventors conceived that mild treatment of cells that preserves the integrity of the membrane such as acid wash, combined with methods of fractionation or separation of the eluted components, results in the isolation of non-membrane proteins that can be induced to translocate to the outer surface of the membrane. The inventors found and report here that:
(1) translocation of Tpm is related to the peak response to stimulation by fibroblast growth factor-2 (FGF-2) stimulation, approximately 6 hours after induction; and
(2) Tpm can be removed from the surface of human umbilical vein ECs (HUVEC) by acid, wash under conditions that preserve integrity of the membrane; and
(3) Vimentin can be removed from the surface of FGF-2 stimulated HUVEC by acid wash under conditions that preserve integrity of the membrane;
(4) A number of other proteins normally appearing in the cytosol can be removed from the surface of HUVEC by acid wash under conditions that preserve integrity of the membrane and;
(5) a similar approach can be applied to other cells that have been stimulated with growth factors or cytokines or cells that are otherwise stressed and therefore not quiescent such as (but not limited to) tumor cells, monocytes/macrophages, neutrophils, T cells and progenitor cells including stem cells.

Other proteins have been shown to be “abnormally” translocated to the cell surface of endothelium under angiogenic conditions. Some of these appear to act as receptors for anti-angiogenic proteins:
(a) the F₁F₀-ATPase subunit which binds angiostatin,
(b) GRP78 (heat shock protein; HSP) which binds anti-angiogenic plasminogen kringle 5 (K5) (see new review for reference) and of course,
(c) Tpm which binds high molecular weight kininogen (HKa), histidine-proline-rich glycoprotein (HPRG), annexins and other HSPs.

According to the present invention, ECs exposed to an angiogenic stimulus “abnormally” translocate proteins to the surface membrane in what the present inventors believe to be a general response of stressed cells. Stress may occur in response to environmental stimuli such as hypoxia or oxidative stress. In addition, a cell that is undergoing proliferation or r differentiation may also be considered to represent a stressed state or phenotype. Thus, ECs and other cells that are exposed to growth factors and cytokines would also translocate cytosolic proteins to the cell surface and are therefore contemplated as part of this invention. In ECs, some of the translocated proteins take on the function of cell-surface receptors for anti-angiogenic molecules. Thus, translocated Tpm becomes a surface receptor for HKa, HPRG and endostatin; translocated F₁F₀ becomes a surface receptor for angiostatin; and translocated GRP78 becomes a surface receptor for plasminogen kringle 5 (K5). According to the present invention, other proteins translocated under similar conditions are involved in regulating other functions that are characteristic of activated ECs. In cells other than ECs, translocated proteins may also regulate key biological functions such as cell survival.

Given the knowledge that many translocated proteins lack a transmembrane domain, and are therefore not anchored, the present inventors conceived of the approach of removing these proteins from the surface of a stressed cell by washing the cells with a buffered, mildly acidic (pH 1.0-5.0), or by protease digestion or membrane isolation. Acid wash is preferred because it permits distinction (i.e., fractionation) between membrane-anchored proteins (not removable by mild acid wash) and translocated proteins which are described herein as not being anchored to the membrane.

According to the present invention, after treating cells to remove non-anchored proteins, a procedure is carried out that compares the pattern of isolated proteins of “stimulated” cells and control (non-stimulated; non-stressed) cells. In this manner, proteins that translocated to the membrane upon the foregoing stimulation can be identified. Furthermore, the presence of cytosolic proteins on the surface of cells could indicate a particular pathology. Since these proteins are not normally found on the outer surface of the cell, these pathologies may be manifest as the presence of abnormal auto-antibodies. As such, those proteins can be targeted for therapy, imaging and diagnosis.

In the case of a translocated protein serving as a receptor for a known ligand, the invention includes evaluating receptor occupancy as an additional method for characterizing the translocated protein, e.g., the appearance of, or concentration of, that particular receptor.

In another embodiment, the translocated proteins may be identified by, for instance, excising them from a gel and submitting the samples to tryptic digestion and mass spectroscopy analysis.

In another embodiment, proteins that associate with acid-extractable proteins and are membrane anchored may be identified by cross-linking and subsequent acid wash and identification by tryptic digestion and mass spectroscopy analysis.

In one embodiment, comparison of the acid wash from ECs stimulated by FGF-2 (or any other angiogenic agent such as VEGF or HGF) to the acid wash of ECs kept in basal media serves as an approach to identifying new proteins that share this property of translocation to the membrane under angiogenic conditions.

In other embodiments, such approaches are used to evaluate (a) ECs activated with pro-inflammatory stimuli such as IL-8 or the induction of NFκB or (b) cells other than ECs stressed by hypoxia, chemotherapeutic agents, etc.

According to this invention, in ECs, externalized proteins provide a survival signal, the displacement of which leads to inhibition of angiogenesis. A similar function is attributed to externalized proteins in cells other than ECs.

According to the present inventors' conception, resistance to chemotherapy by certain agents is regulated to some degree, by externalized (translocated) tumor cell proteins (or proteins of non-tumor cells, i.e., normal, cells) of a host being treated with a chemotherapeutic agent. These molecules are identified using the acid wash, proteolysis and other methods discussed herein.

Such translocated, removed and, optionally, isolated molecules are conceived herein as being novel targets for attenuating tumor resistance.

The present invention is also directed to a method for identifying ligands that (a) are found exclusively in a form bound to their receptors or (b) found at relatively higher levels in cells subjected to an angiogenic environment such as that created by tumor cells and in tumor tissue.

Samples from angiogenic and from “control” non-angiogenic ECs (or similarly, from stressed cells and from control cells kept under non-stressed conditions) are analyzed using any analytic methods available that would permit identification and characterization of such removed molecules. Examples include 2D-gel electrophoresis or mass spectrometry to identify proteins selectively present on the cell surface under a particular set of conditions. Further, the proteins of interest are identified and may be sequenced using standard techniques, coupled with amino acid analysis and sequencing

Once such proteins have been identified, they may be isolated and used in screening assay. They may be used to induce antibodies that may be specific for the translocated form of the protein, so that such antibodies are useful to identify cells that have undergone this translocation process. Depending on the cell type, location, etc., such an antibody based method could be useful for diagnosing or prognosing conditions in which the cells are being stressed or stimulated in an angiogenic environment, the presence of which is not otherwise detectable. This may serve a diagnostic or prognostic test for a number of pathophysiological processes.

The differences in patterns of translocated proteins between stimulated or stressed cells and quiescent cells may also represent a diagnostic method. For example, alterations in the proteome of a cell may identify it as an angiogenic endothelial cells or a tumor cell. In patients, knowledge of the alterations in translocated proteins may allow measurements of these proteins in the plasma or in disease tissue, for example in a tumor biopsy. The presence of certain of these proteins in a patient may allow the diagnosis of a particular disease or could lead to a prognosis for example being able to stage a tumor or predict whether a cancer patient will require adjuvant therapy.

ECs treated with FGF-2, a known angiogenic stimulus, are compared with ECs kept in control medium supplemented with 2% serum. Inspection of 2D gels of membrane preparation revealed several distinct spots present on the cells that have been exposed to FGF-2 but are not detectable on control cells. These proteins may serve as novel targets for use in therapy, imaging, targeting etc.

Identification of novel targets for therapeutic intervention, targeting and/or imaging is fundamental to approaches against many diseases. The present description exemplifies and focuses on how this may be accomplished for angiogenesis, so that the present approach is applicable target identification for any of a large number of disease states wherein angiogenesis is a pathogenetic component, e.g., cancer, rheumatoid arthritis and a number of ocular disorders. Anti-angiogenic therapy focused on such newly discovered targets may have a significant impact in the treatment of such diseases.

Many methods have been described for the purpose of comparing quiescent versus stimulated cells at a proteomic or genomic level. The method described here is highly advantageous in that (a) it is much simpler and (b) it concentrates on targets on the cell membrane—targets that are the most accessible to drugs or biologics and are thereby the most desirable to select for development of drugs, imaging agents or targeting agents.

Another aspect of the invention relies on the fact that cells under stress will translocate proteins to the membrane that are not present when the cells are in a quiescent state. The methods described herein coupled with conventional techniques not only permit comparisons of protein expression patterns under stress and quiescent (i.e., non-stress) conditions but also provide a basis for removing and separating those proteins. Such abnormally translocated proteins may be removed from the cell surface in a number of ways while preserving membrane integrity. These include, but are not limited to, membrane isolation, proteolysis, high salt treatment and metal chelation. Moreover, any of a number of methods may be used to distinguish the pattern of cell surface proteins before and after their removal, and thereby aid in target identification.

Proteins that are only translocated and appear on the surface of stressed cells will also provide selectivity if these proteins are targeted therapeutically since they will not be present on the surface of normal cells.

Having now generally described the invention, the same will be more readily understood through reference to the following examples which are provided by way of illustration, and are not intended to be limiting of the present invention, unless specified.

### EXAMPLE 1

### Angiogenic Stimulus, FGF-2, Induces Changes in Profile of Membrane Proteins in HUVEC

HUVECs purchased from Cascade Biologicals were grown in M200 medium supplemented with LSGS which includes 2% fetal bovine serum (FBS). HUVECs were starved by overnight culture in M200 supplemented with 2% FBS. The following day, cells were treated with the indicated amounts of FGF-2 (or control buffer), washed and treated with 50 mM glycine, 150 mM NaCl, pH 3.0 for three minutes (“acid wash”).

The supernatant was transferred to a tube containing IM Tris buffer, pH 7.5 in sufficient amount to bring the pH of the mixture to pH ˜7.0. The supernatant was concentrated and precipitated with acetone. The pellet was re-suspended in SDS buffer and subjected to polyacrylamide gel electrophoresis (PAGE).

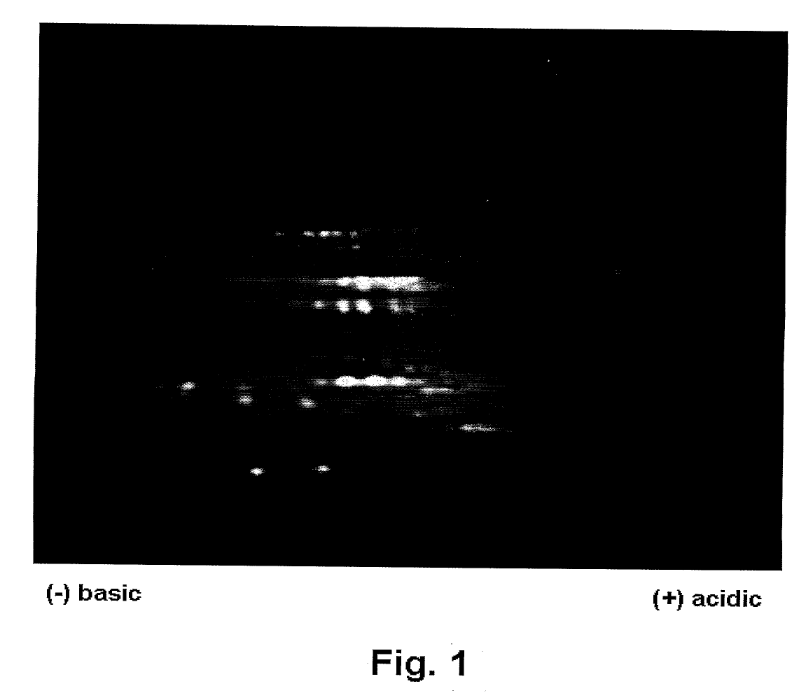

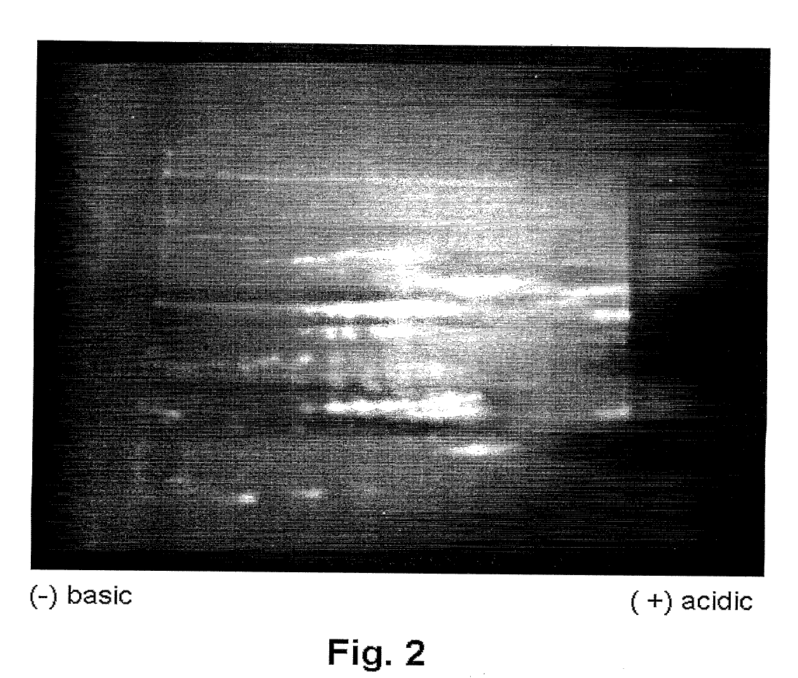

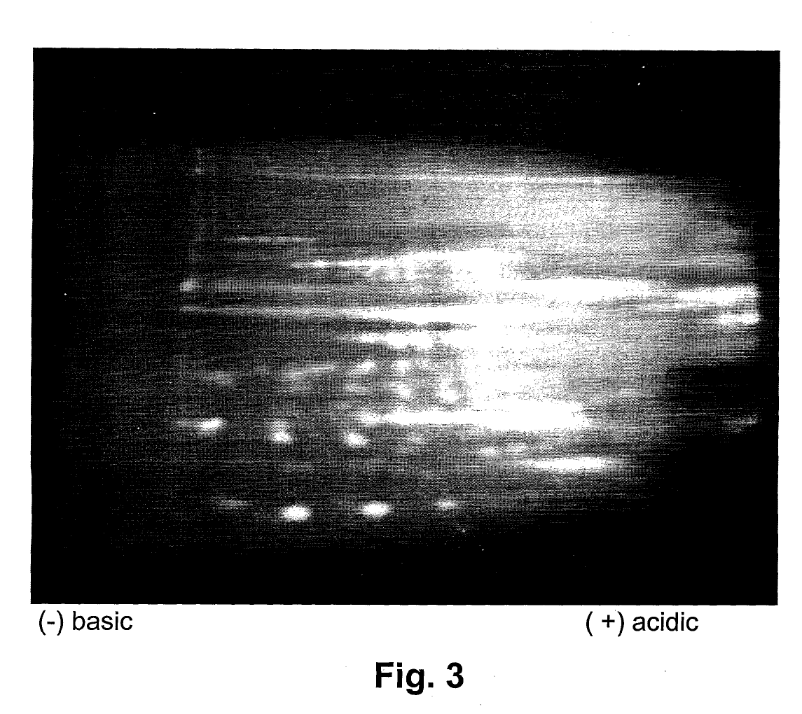

The two treatment groups±FGF-2 showed a different pattern of proteins detected after acid wash and resolution by 2D electrophoresis shown in FIGS. 1-3.

### EXAMPLE 2

### Angiogenic Stimulus, FGF-2, Stimulates Translocation of Tropomyosin without Damaging Cell Membrane

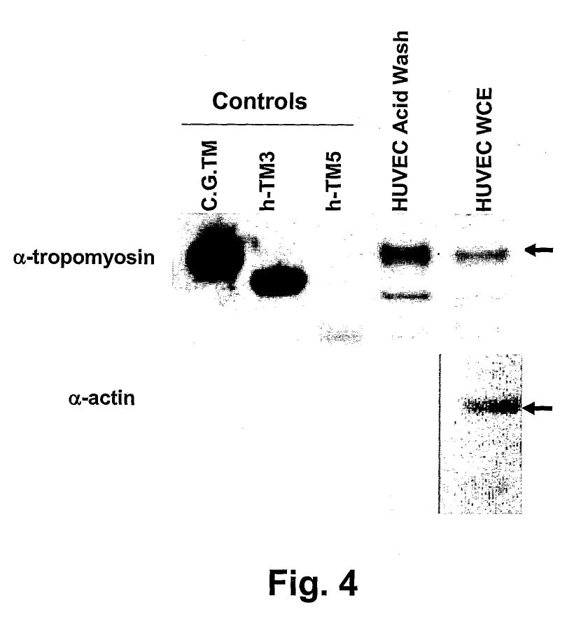

As noted above, TPM translocated to the surface of ECs under angiogenic conditions (Donate et al., Proc. Natl. Acad. Sci. U.S.A. 99:12224-12229, 2002). Using the methods described herein (see Example I for acid wash procedure. Tpm was removed from the surface of HUVECs while preserving the integrity of the membrane, as shown in FIGS. 4-8. The conclusion that membrane integrity was not compromised was based on the finding that cytosolic proteins were not released under the same conditions.

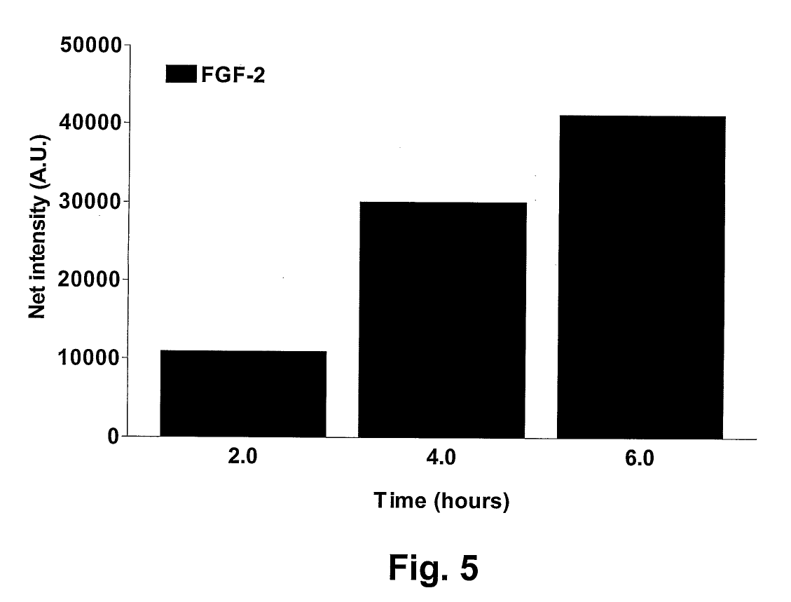

Results shown in FIG. 5 demonstrate that translocation of Tpm in HUVEC was dependent on FGF-2 stimulation, and peaked at approximately 6 hours post induction.

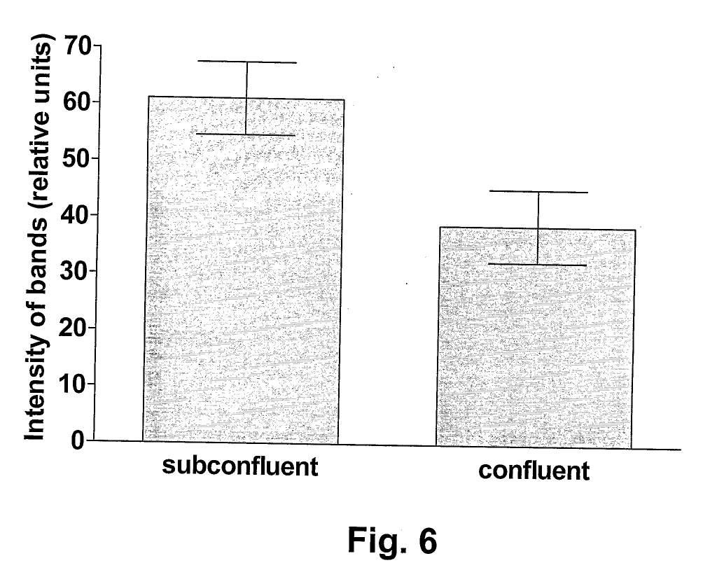

Results shown in FIG. 6 demonstrate that translocation of Tpm occurs to larger extent in non-confluent, proliferating HUVEC than in confluent, more quiescent cells. This result also argues that the origin of the Tpm on the surface of HUVEC is not from cell lysis, since the levels of Tpm should have been equal, or even greater, in confluent cells that have been in culture longer than sub-confluent cells.

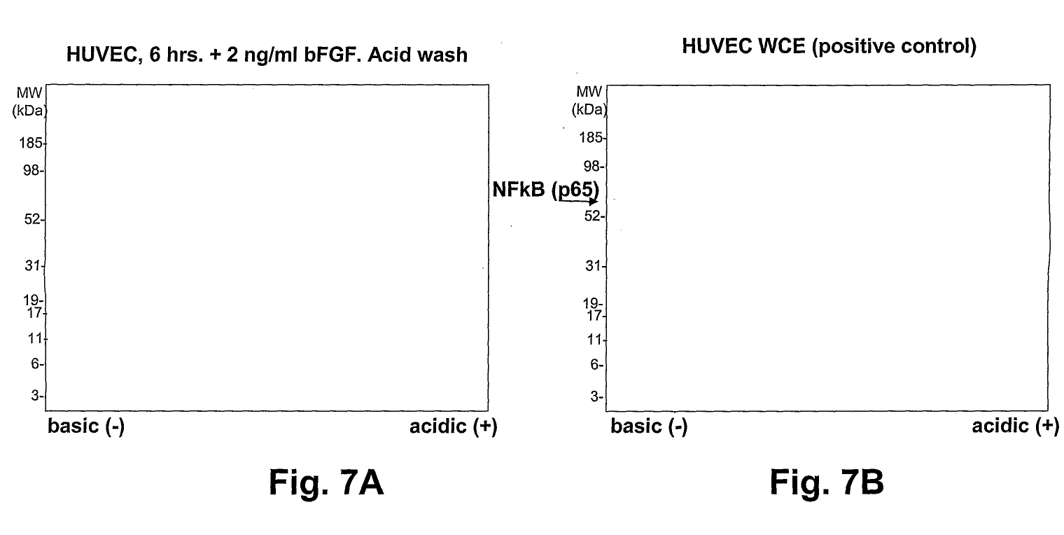

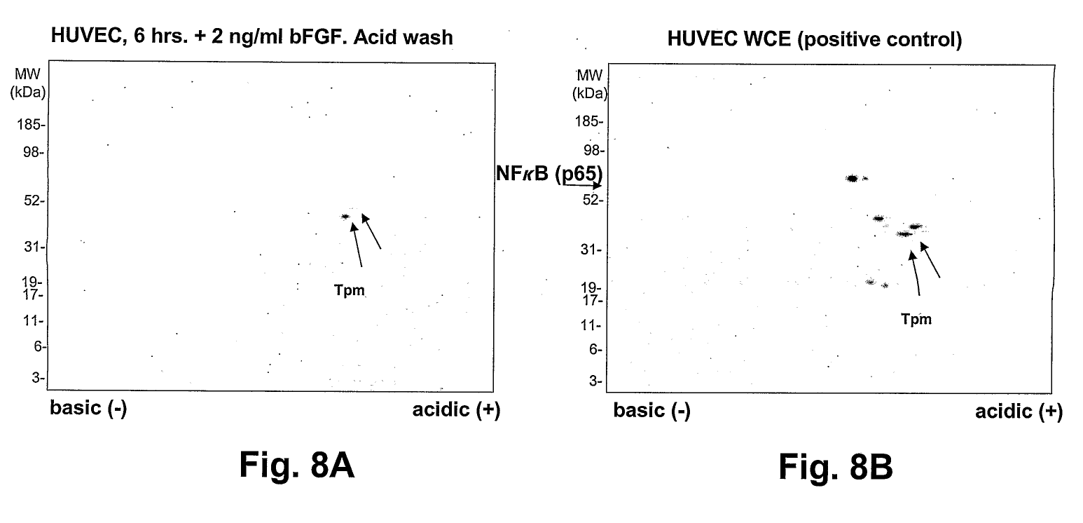

As shown in FIGS. 4 and 7A/7B, neither actin nor NFκB, both abundant cytosolic proteins, were detectable on blots of acid-wash proteins from these using the appropriate antibodies. However, Tpm was detected (FIG. 8A/8B). Using HUVECs that were subjected to an acid wash, followed by acetone precipitation, Western blots with polyclonal rabbit anti-Tpm antibody or a anti-Tpm mAb detected Tpm. A specific antibody against actin was used for similar analysis.

### EXAMPLE 3

### Angiogenic Stimulus, FGF-2, Stimulates Translocation of Vimentin without Damaging Cell Membrane

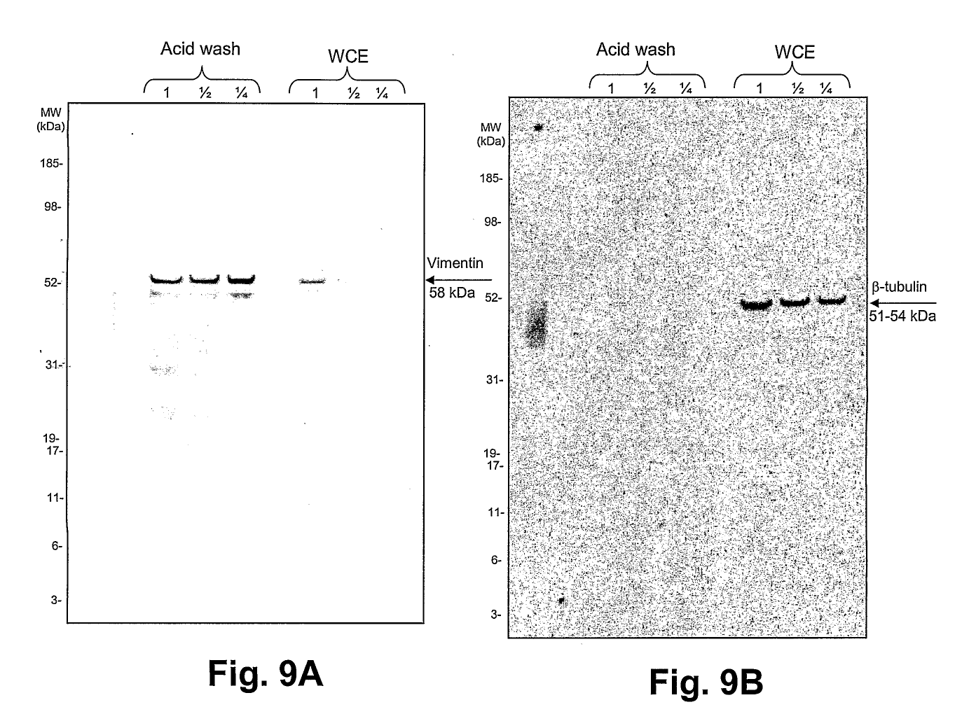

Using HUVECs that were subjected to an acid wash (see Example I for acid wash procedure), followed by acetone precipitation, Western blots with polyclonal goat anti-vimentin antibody detected vimentin. A specific antibody against β-tubulin was used for similar analysis. Vimentin was removed from the surface of HUVEC while preserving the integrity of the membrane, as shown in FIGS. 9a/9b and was shown before in FIGS. 4 and 7. The conclusion that membrane integrity was not compromised was based on the finding that an abundant cytosolic protein, β-tubulin, was not released under the same conditions.

### EXAMPLE 4

### Detection of Proteins in Acid Wash Fraction of HUVEC Using Tryptic Peptides, Mass Spectroscopy 9(MS/MS and MALDI-TOF) and Identification of Protein Sequences in NCBI Database

HUVEC were stimulated with FGF-2 and subjected to an acid wash as described above. The fraction was concentrated and subjected to denaturation and trypsin digestion. The resulting peptides were analyzed by Mass Spectrometry (MS). An aliquot was used to generate a peptide mass fingerprint (PMF) by matrix-assisted laser-desorption ionization time-of-flight mass spectrometer (MALDI TOF-TOF, 4700 proteomics analyzer, Applied Biosystems) and used to verify the extent of the digestion.

The rest of the sample was analyzed by liquid chromatography/mass spectroscopy (LC MS/MS. In this approach the peptides are sequentially eluted of a C18 RP HPLC column with an increased gradient of acetonitrile and analyzed by on-line by tandem MS on a ion iclotron Fourier Transform mass spectrometer (LTQ-FT, ThermoFinnigan). The high mass accuracy of this instrument (better that 2 ppm), in combination with the high sensitivity and specificity of the resulting MS/MS spectra, is sufficient to characterize proteins from completely sequenced genomes.

Protein identification was done by comparing the MS and MS/MS spectra with the predicted masses and fragmentation patterns of proteins in protein databases or DNA databases (after translation of open reading frames) databases, including EST databases. It should be noted that this is an exquisitely sensitive approach for identifying the constituents of complex mixtures of proteins. Non-redundant protein databases now have around 2,000,000 entries (the NCBI protein data based contained 2286058 entries as of June 2005).

In the present case, the data was compared with proteins in the NCBI Database, utilizing the SEQUEST algorithm. Hits are shown below in Table 1. It is clear that a large number of proteins, many of which are potential useful targets for drug development, were found in the acid wash fraction. It is noteworthy that tropomyosin isoform 3 and TC22 (similar to TM5) appeared in this list; in agreement with the results above showing that tropomyosin is externalized in FGF-2-treated endothelial cells.

TABLE 1
Proteins from NCBI Database Identified in Acid Wash Fraction of HUVECs Stimulated with bFGF-2

| | | | NCBI | | |
|---|---|---|---|---|---|
| | | MOWSE | Accession | Peptides | |
| | Reference | Score | # | (Hits) | |
| #1 | bA255A11.8 (novel protein similar to annexin A2 (ANXA2) | 674.3 | 12314197.0 | 68 | (66 1 1 0 0) |
| | (lipocortin II, | | | | |
| #2 | Annexin A2 [Homo sapiens] | 300.3 | 16306978.0 | 30 | (30 0 0 0 0) |
| #3 | annexin 5; endonexin II; anchorin CII; lipocortin V; placental | 240.3 | 4502107.0 | 24 | (24 0 0 0 0) |
| | anticoag | | | | |
| #4 | alpha enolase [Homo sapiens] | 220.4 | 2661039.0 | 22 | (22 0 0 0 0) |
| #5 | vimentin [Homo sapiens] | 188.2 | 5030431.0 | 19 | (18 1 0 0 0) |
| #6 | eukaryotic translation elongation factor 1 alpha 1; CTCL tumor | 160.3 | 4503471.0 | 16 | (16 0 0 0 0) |
| | antigen; | | | | |
| #7 | mutant beta-actin (beta′-actin) [Homo sapiens] | 96.4 | 28336.0 | 10 | (8 2 0 0 0) |
| #8 | peptidylprolyl isomerase A; cyclophilin A [Mus musculus] | 90.3 | 6679439.0 | 9 | (9 0 0 0 0) |
| #9 | protease, serine, 11 [Homo sapiens] | 90.2 | 4506141.0 | 9 | (9 0 0 0 0) |
| #10 | annexin I; annexin I (lipocortin I); lipocortin I [Homo sapiens] | 80.3 | 4502101.0 | 8 | (8 0 0 0 0) |
| #11 | peroxiredoxin 1; natural killer-enhancing factor A; proliferation- | 70.2 | 4505591.0 | 7 | (7 0 0 0 0) |
| | assoc | | | | |
| #12 | A Chain A, Human Plasminogen Activator Inhibitor Type-1 In | 60.2 | 4699714.0 | 6 | (6 0 0 0 0) |
| | Complex With A Penta | | | | |
| #13 | PPIB_HUMAN Peptidyl-prolyl cis-trans isomerase B precursor | 60.2 | 118090.0 | 6 | (6 0 0 0 0) |
| | (PPlase) (Rotamase) | | | | |
| #14 | envelope glycoprotein [Human immunodeficiency virus type 1] | 52.4 | 11875486.0 | 7 | (0 5 2 0 0) |
| #15 | ribosomal protein S19 [Homo sapiens] | 50.2 | 16924231.0 | 5 | (5 0 0 0 0) |
| #16 | similar to Spir-1 protein [Homo sapiens] | 44.3 | 37545113.0 | 6 | (2 1 2 1 0) |
| #17 | Homo sapiens S100 calcium binding protein A6 (calcyclin) | 40.3 | 30584467.0 | 4 | (4 0 0 0 0) |
| | [synthetic cons | | | | |
| #18 | activated T-cell marker CD109 [Homo sapiens] | 40.3 | 37359236.0 | 4 | (4 0 0 0 0) |
| #19 | enolase 1; phosphopyruvate hydratase; MYC promoter-binding | 40.2 | 4503571.0 | 4 | (4 0 0 0 0) |
| | protein 1; n | | | | |
| #20 | lactate dehydrogenase, LDH-A [human, Peptide Partial Mutant, | 40.1 | 237993.0 | 4 | (4 0 0 0 0) |
| | 10 aa] | | | | |
| #21 | killer cell immunoglobulin-like receptor precursor [Homo | 38.2 | 12006229.0 | 5 | (2 2 0 0 1) |
| | sapiens] | | | | |
| #24 | envelope protein [Human immunodeficiency virus type 1] | 34.2 | 10732710.0 | 4 | (2 1 1 0 0) |
| #25 | H1 histone family, member 5 [Homo sapiens] | 34.2 | 4885381.0 | 4 | (3 0 0 1 0) |
| #29 | Homo sapiens profilin 1 [synthetic construct] | 30.4 | 30584265.0 | 3 | (3 0 0 0 0) |
| #30 | Crystal Structure Of Human Recombinant Procathepsin B At 3.2 | 30.3 | 2982114.0 | 3 | (3 0 0 0 0) |
| | Angstrom Resolut | | | | |
| #31 | peroxiredoxin 2 isoform a; thioredoxin-dependent peroxide | 30.3 | 32189392.0 | 3 | (3 0 0 0 0) |
| | reductase 1; | | | | |
| #32 | Homo sapiens lactate dehydrogenase A [synthetic construct] | 30.2 | 30584487.0 | 3 | (3 0 0 0 0) |
| #33 | GDP dissociation inhibitor 1; mental retardation, X-linked 41; | 30.2 | 4503971.0 | 3 | (3 0 0 0 0) |
| | mental r | | | | |
| #34 | transgelin 2; SM22-alpha homolog [Homo sapiens] | 30.2 | 4507357.0 | 3 | (3 0 0 0 0) |
| #35 | H1 histone family, member 3; histone H1c [Homo sapiens] | 30.2 | 4885377.0 | 3 | (3 0 0 0 0) |
| #36 | thioredoxin peroxidase; thioredoxin peroxidase (antioxidant | 30.2 | 5453549.0 | 3 | (3 0 0 0 0) |
| | enzyme) [Ho | | | | |
| #37 | similar to dJ680N4.2 (ubiquitin-conjugating enzyme E2D 3 | 30.1 | 27485630.0 | 3 | (3 0 0 0 0) |
| | (homologous t | | | | |
| #38 | ubiquitin-conjugating enzyme E2D 2 isoform 2; ubiquitin carrier | 30.1 | 33188456.0 | 3 | (3 0 0 0 0) |
| | protei | | | | |
| #54 | L protein [Human parainfluenza virus 3] | 22.2 | 1255659.0 | 3 | (2 0 0 0 1) |
| #58 | similar to Glyceraldehyde 3-phosphate dehydrogenase, liver | 20.3 | 37551788.0 | 2 | (2 0 0 0 0) |
| | (GAPDH) [Ho | | | | |
| #40 | similar to cytoplasmic beta-actin [Homo sapiens] | 28.5 | 29736622.0 | 3 | (2 1 0 0 0) |
| #62 | tropomyosin 3 [Homo sapiens] | 20.2 | 22748619.0 | 2 | (2 0 0 0 0) |
| #63 | PRO1708 [Homo sapiens] | 20.2 | 7959791.0 | 2 | (2 0 0 0 0) |
| #64 | tropomyosin isoform TC22 [Homo sapiens] | 20.2 | 9508585.0 | 2 | (2 0 0 0 0) |
| #65 | phosphoglycerate kinase 1 [Homo sapiens] | 20.2 | 4505763.0 | 2 | (2 0 0 0 0) |
| #66 | cofilin 1 (non-muscle) [Homo sapiens] | 20.2 | 5031635.0 | 2 | (2 0 0 0 0) |
| #67 | A Chain A, Crystal Structure Of Truncated Human Rhogdi | 20.2 | 14278159.0 | 2 | (2 0 0 0 0) |
| | Quadruple Mutant | | | | |
| #68 | KIAA0803 protein [Homo sapiens] | 20.2 | 3882327.0 | 2 | (2 0 0 0 0) |
| #69 | A Chain A, Macrophage Migration Inhibitory Factor (Mif) | 20.2 | 13399777.0 | 2 | (2 0 0 0 0) |
| | Complexed With Inhibit | | | | |
| #70 | KPY2_HUMAN Pyruvate kinase, M2 isozyme | 20.2 | 125604.0 | 2 | (2 0 0 0 0) |
| #71 | nucleoside-diphosphate kinase 1 isoform a [Homo sapiens] | 20.2 | 38045913.0 | 2 | (2 0 0 0 0) |
| #72 | plastin 3; T isoform [Homo sapiens] | 20.2 | 7549809.0 | 2 | (2 0 0 0 0) |
| #73 | Aldolase A (E.C.4.1.2.13) | 20.2 | 229674.0 | 2 | (2 0 0 0 0) |
| #74 | TALDO1 protein [Homo sapiens] | 20.2 | 17511894.0 | 2 | (2 0 0 0 0) |
| #75 | UEV1Bs [Homo sapiens] | 20.1 | 2689608.0 | 2 | (2 0 0 0 0) |
| #76 | glucosamine (N-acetyl)-6-sulfatase precursor; N- | 20.1 | 4504061.0 | 2 | (2 0 0 0 0) |
| | acetylglucosamine-6-sul | | | | |
| #77 | lipin 1 [Homo sapiens] | 20.1 | 22027648.0 | 2 | (2 0 0 0 0) |

All the references cited above are incorporated herein by reference in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A method of detecting or identifying one or more non-membrane proteins that translocate to the surface of stressed or stimulated cells but are not present on the surface of the same cells when these cells are quiescent, the method comprising
(a) treating cells from a biological sample under conditions that remove the translocated protein from the cell surface but preserve the integrity of the cells' membrane,
(b) detecting or identifying proteins removed from the cells surface, thereby detecting or identifying said translocated protein.

2. The method of claim 1 wherein step (a) results in removal from the cells of non-anchored proteins.

3. The method of claim 1 wherein said removing of step (a) is accomplished by acid wash under isotonic conditions to minimize cell lysis using a buffer with a pH range of 1 to 5.

4. The method of claim 1 wherein said removing of step (a) is accomplished by membrane isolation, proteolysis, high salt treatment or metal chelation.

5. The method of claim 1 wherein the translocated protein is a receptor for a known ligand and the detecting or identifying comprises, prior to step (a), measuring occupancy of said receptors on the surface of said cells using said known ligands in a receptor-ligand binding assay.

6. The method of claim 1 wherein step (b) comprises a step of comparing proteins removed from the stressed cells with proteins removed under the same conditions from control cells of the same or similar cell type that are not stimulated or stressed.

7. The method of claim 6 wherein said comparison is between cells stressed by an angiogenic agent and control cells maintained in a basal medium.

8. The method of claim 1 wherein the translocated proteins are cytosolic proteins.

9. The method of claim 1 wherein said translocated and removed proteins are identified by gel electrophoresis or by two-dimensional (2D) analysis using gel electrophoresis and isoelectric focusing.

10. (canceled)

11. The method of claim 9 wherein the translocated and removed proteins are further identified by excision of a spot or spots from 2D gels followed by
(a) mass spectroscopy; or
(b) proteolytic digestion and mass spectroscopy; or
(b) tryptic digestion western blotting and
(i) N-terminal sequencing or
(ii) amino acid composition analysis.

12-13. (canceled)

14. The method of claim 1 wherein said translocated proteins are crosslinked prior to the removing step and are identified by proteolytic digestion and mass spectroscopy.

15.-16. (canceled)

17. The method of claim 1 wherein the stressed cells are stressed by an environmental stimulus.

18. (canceled)

19. The method of claim 1 wherein the stressed cells are cells undergoing proliferation or differentiation.

20. The method of claim 1 wherein the stressed cells are stressed by exposure to a growth factor or cytokine.

21. The method of claim 1 wherein the cells are stressed as a result of exposure to a pro-inflammatory stimulus or a stimulus that induces NFκB.

22. (canceled)

23. The method of claim 1 wherein the cells are stressed as a result of exposure to a cancer chemotherapeutic drug or agent.

24. The method of claim 1 wherein the cells are endothelial cells

25. The method of claim 24 wherein the translocated and removed protein is tropomyosin or vimentin.

26. The method of claim 24 wherein step (a) is conducted about six hours after initiation of cells are stressed or stimulated.

27. (canceled)

28. The method of claim 1 wherein the stressed cells are tumor cells, monocyte/macrophages, neutrophils, T lymphocytes, or stem or progenitor cells.

29. (canceled)

30. The method of claim 1 wherein the translocated protein serves as a receptor for an anti-angiogenic protein.

31. The method of claim 30 wherein
(a) the anti-angiogenic protein is angiostatin, plasminogen kringle region 5 (K5) high molecular weight kininogen (HKa) histidine-proline-rich glycoprotein (HPRG) or an annexin; and
(b) the translocated protein that binds to the anti-angiogenic protein is:
(i) in the case of angiostatin, F₁F₀-ATPase subunit
(ii) in the case of K5 GRP78 or
(iii) in the case of HPRG or the annexin, tropomyosin.

32.-36. (canceled)

37. A method of determining whether tumor cells or normal cells of a subject with a tumor are resistant to a chemotherapeutic agent which results in translocation or externalization of internal proteins to the cell surface, which method comprises:
(i) subjecting the subject or tumor cells or normal cells from the subject to chemotherapy with said agent and
(ii) detecting or identifying proteins removed from the cells surface with the method of claim 1,
wherein the detection of the presence of said translocated proteins is indicative of resistance to chemotherapy by said agent.

38. The method of claim 1 further comprising, after detecting or identifying said translocated protein, a step of (c) isolating said protein.

39. The method of producing an antibody specific for a translocated protein as the protein is expressed on the surface of a stressed cell, comprising performing the method of claim 38 to isolate said protein, and further, the step of
(d) producing an antibody specific for said translocated isolated protein that distinguishes between said translocated protein and the same protein that has not been translocated by said stress.

40. A method for identifying cells that have undergone stress-related translocation of internal proteins to the cell surface comprising contacting cells suspected of having such a translocated protein on its surface with an antibody produced in accordance with claim 39.

41. The method of claim 38 wherein the cells are stressed or stimulated by an angiogenic stimulus that results in the presence on the cells' surfaces of said translocated protein.

42. A method of diagnosing a pathological process in a subject in whom cells are stressed or stimulated to translocate internal proteins to the cell surface, comprising
(a) detecting or identifying said translocated proteins on a sample of cells from said subject; and
(b) comparing said translocated proteins detected or identified in (a) with control, quiescent cells from the same subject or a control subject not undergoing said pathological process.

43. The method of claim 42 wherein said pathological process in an angiogenic process.
